(19) United States
(12) Patent Application Publication
(10) Pub. No.: US 2026/0353894 A1
(43) Pub. Date: Nov. 5, 2026

(54) WATERPROOF ULTRAVIOLET LIGHT-EMITTING MODULE

(71) Applicant: STANLEY ELECTRIC CO., LTD., Meguro-ku, Tokyo (JP)

(72) Inventor: Masahiro AKIMOTO, Tokyo (JP)

(73) Assignee: STANLEY ELECTRIC CO., LTD., Meguro-ku, Tokyo (JP)

(21) Appl. No.: 19/485,010

(22) PCT Filed: Apr. 30, 2024
(86) PCT No.: PCT/JP2024/016650

§ 371 Date: Nov. 17, 2025

(30) Foreign Application Priority Data
May 18, 2023 (JP) 2023-082147

(51) Int. Cl. H10H 29/85; A61L 2/104; H10H 20/85; H10H 20/854; H10H 20/855; H10H 20/857; H10H 20/858; H10H 29/854; H10H 29/855

(52) U.S. Cl. CPC H10H 29/8506; H10H 20/8506; H10H 20/8508; H10H 20/854; H10H 20/855; H10H 20/857; H10H 20/8582; H10H 20/8583; H10H 29/8508; H10H 29/854; H10H 29/855; H10H 29/857; H10H 29/8582; H10H 29/8583; A61L 2/104

(57) ABSTRACT

A waterproof ultraviolet light-emitting module is capable of preventing deterioration in an LED die due to moisture and preventing deterioration in the LED die due to heat. An ultraviolet light LED package is mounted on a mounting substrate. A lens includes a lens body having a dome shape and a flange portion. The flange portion of the lens is interposed between a cover and a housing, and the cover is pressed against and fixed to the housing by a fastening tool. The housing has a convex rib provided thereon. The lower surface of the flange portion of the lens is deformed by being pressed against the rib such that a space in the lens is sealed.

### TECHNICAL FIELD

The present invention relates to a light-emitting module that emits ultraviolet light.

### BACKGROUND ART

Known is an apparatus that sterilizes fluids such as water and air by irradiating the fluids with ultraviolet light.

For example, PTL 1 discloses an apparatus configured such that a transparent tube is spirally wound and is disposed inside a housing, water flows through the tube, and a semiconductor light-emitting element disposed on the inner wall of the housing irradiates the water flowing through the tube with ultraviolet light to sterilize the water. A fluorine-based resin or a silicone-based resin is used as a material for the tube.

Further, PTL 2 discloses a light-emitting unit configured to emit ultraviolet light and disposed inside a duct of a vehicle air conditioner. Here, the light-emitting unit irradiates air flowing through the inside of the duct with ultraviolet light. The light-emitting unit has a structure including a plurality of ultraviolet light-emitting elements fixedly arranged around the periphery of a support plate connected to a heat sink and a heat dissipation fin.

Meanwhile, it is known that the semiconductor light-emitting element that emits ultraviolet light has a property that a material of the semiconductor light-emitting element easily deteriorates due to moisture as compared with light-emitting elements that emit light of other wavelengths.

PTL 3 discloses, as a light-emitting module having a waterproof structure, a structure in which a recessed portion is provided in a housing made of a resin, a packaged light-emitting element is disposed inside the recessed portion, and the light-emitting element is covered with a lens having a space provided therein. It is described that, as the light-emitting element, a light-emitting element having a wavelength of ultraviolet light can be used.

### CITATION LIST

### Patent Literature

PTL 1: JP2018-148938A
PTL 2: JP2022-113426A
PTL 3: JP2022-125391A

### SUMMARY OF INVENTION

### Technical Problem

PTLs 1 and 2 do not describe a waterproof structure of a light-emitting element that emits ultraviolet light.

On the other hand, although PTL 3 discloses a light-emitting module having a waterproof structure implemented by covering a light source with a housing made of a resin and a lens, there is no description about a specific structure regarding a waterproof structure of a connection portion between the lens and the housing.

Since a light-emitting element that emits ultraviolet light is a material that easily deteriorates due to moisture as compared with light-emitting elements having other wavelengths, it is crucial to provide a waterproof structure for the connection portion between the lens and the housing. However, since ultraviolet light causes deterioration in resin, a waterproof structure made of a resin, such as an O-ring, cannot be used.

Furthermore, the light-emitting element that emits ultraviolet light generates a larger amount of heat than those of light-emitting elements having other wavelengths. In this case, when a space around the periphery of the light-emitting element is sealed to achieve a waterproof structure, it becomes difficult to dissipate heat into the air.

An object of the present invention is to provide a waterproof ultraviolet light-emitting module capable of preventing deterioration in an LED die due to moisture and preventing deterioration in the LED die due to heat.

### Solution to Problem

To achieve the above-described object, a waterproof ultraviolet light-emitting module of the present invention includes an ultraviolet light source, a mounting substrate, a housing, a lens, a cover, and a fastening tool. The ultraviolet light source is an LED package configured such that an LED die emitting ultraviolet light is enclosed in a package. The mounting substrate includes wiring, is configured such that the ultraviolet light source is mounted thereon, and is disposed on the housing. The housing has a plate shape allowing the mounting substrate to be mounted thereon. The lens is formed of a material allowing the ultraviolet light to pass therethrough and includes a dome-shaped lens body disposed to cover the ultraviolet light source with a space interposed therebetween and a flange portion provided to be integrated with a peripheral edge portion of the lens body. The cover covers the flange portion of the lens. The fastening tool presses the cover against the housing such that the cover is fixed to the housing in a state in which the flange portion of the lens is interposed between the cover and the housing. The housing has a convex rib provided on a portion thereof in contact with the flange portion of the lens, and the convex rib is formed along a peripheral edge of the mounting substrate. A portion of a lower surface of the flange portion of the lens, which is in contact with the rib, is deformed by being pressed against the rib by the cover such that the space in the lens is sealed.

### Advantageous Effects of Invention

According to the present invention, it is possible to provide a waterproof ultraviolet light-emitting module having high water resistance and heat resistance.

### BRIEF DESCRIPTION OF DRAWINGS

FIG. 1 is a perspective view of a waterproof ultraviolet light-emitting module 1 according to an embodiment of the present invention.

FIG. 2 is a cross-sectional view taken along line A-A of the waterproof ultraviolet light-emitting module 1 according to the embodiment.

FIG. 3 is a cutaway perspective view of the waterproof ultraviolet light-emitting module 1 according to the embodiment.

FIG. 4 is a perspective view of each component forming the waterproof ultraviolet light-emitting module 1 according to the embodiment.

FIG. 5 is a cross-sectional view illustrating a structure of an ultraviolet light source (an LED package) of the waterproof ultraviolet light-emitting module 1 according to the embodiment.

FIG. 6 is a diagram illustrating the waterproof and moisture-proof properties of the waterproof ultraviolet light-emitting module 1 according to the embodiment.

FIG. 7 is a cutaway perspective view illustrating an assembly process of the waterproof ultraviolet light-emitting module 1 according to the embodiment.

FIGS. 8(a) to 8(c) are perspective views of the waterproof ultraviolet light-emitting module 1 according to modifications of the embodiment.

### DESCRIPTION OF EMBODIMENTS

An embodiment of the present invention is described below.

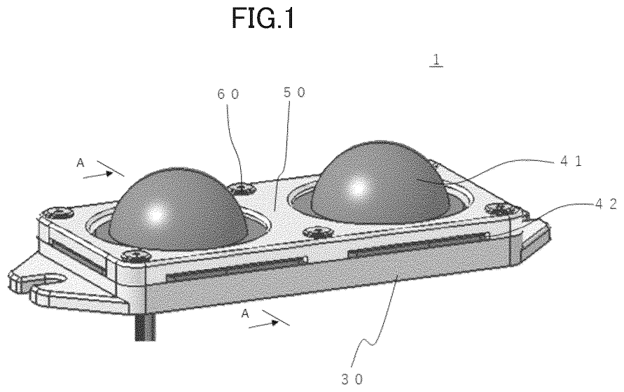

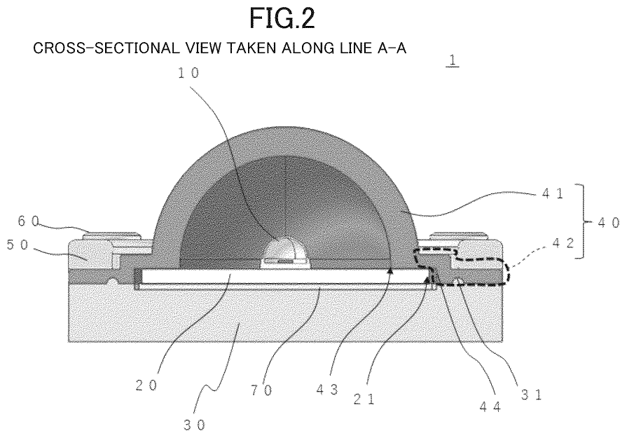

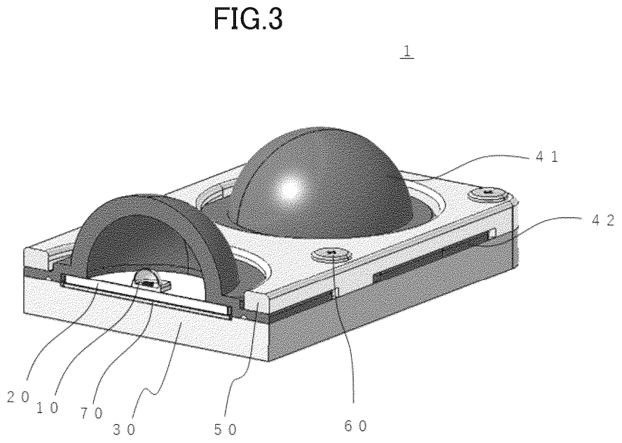

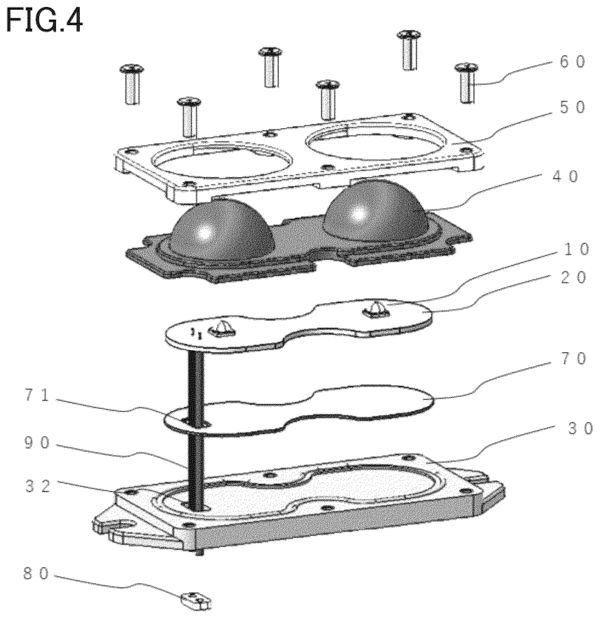

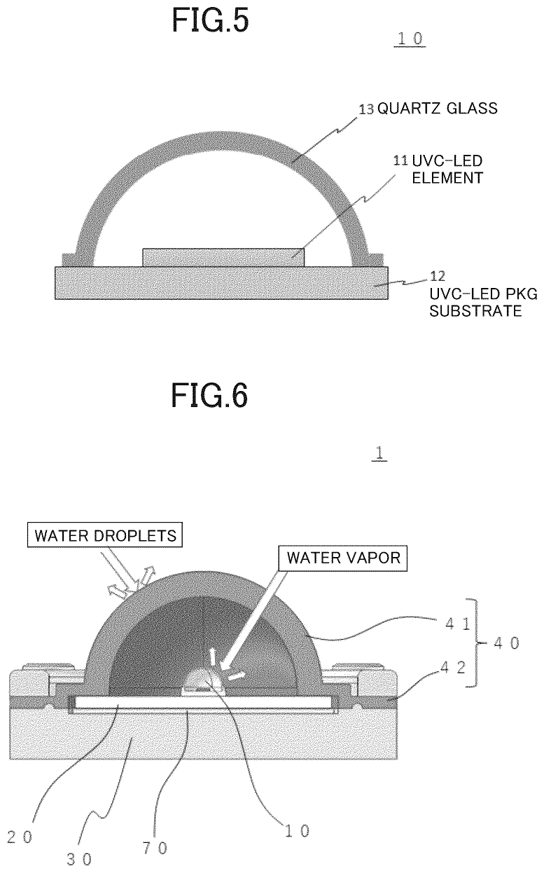

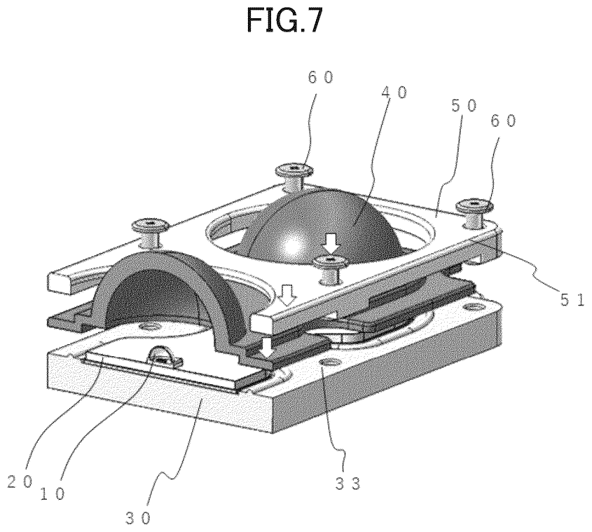

A waterproof ultraviolet light-emitting module 1 according to the embodiment will be described using FIGS. 1 to 7. FIGS. 1 to 3 are a perspective view, a cross-sectional view taken along line A-A, and a cutaway perspective view of the waterproof ultraviolet light-emitting module 1, respectively. FIG. 4 is a perspective view of each component forming the waterproof ultraviolet light-emitting module 1. FIG. 5 is a diagram illustrating a structure of an ultraviolet light source (an LED package) of the waterproof ultraviolet light-emitting module 1. FIG. 6 is a diagram illustrating waterproof and moisture proof, and FIG. 7 is a diagram illustrating an assembly process.

### Configuration of Waterproof Ultraviolet Light-Emitting Module 1

First, a configuration of the waterproof ultraviolet light-emitting module 1 will be described.

As illustrated in FIGS. 1 to 4, the waterproof ultraviolet light-emitting module 1 includes an ultraviolet light source 10, a mounting substrate 20, a metal housing 30, a lens 40, a cover 50, and a fastening tool 60.

### Ultraviolet Light Source (Ultraviolet Light LED Package) 10

As illustrated in FIG. 5, the ultraviolet light source 10 is an LED package configured such that an LED die 11 that emits ultraviolet light is mounted on a package substrate 12 made of ceramic or the like, and a space around the periphery of the LED die 11 mounted on the package substrate 12 is sealed with a sealing member 13 made of quartz glass having a hemispherical shape or the like. Hereinafter, the ultraviolet light source 10 will be referred to as an ultraviolet light LED package 10.

Although not illustrated in FIG. 5, the package substrate 12 is provided with wiring such as a via for supplying power to the LED die 11 and is connected to an electrode of the LED die 11 by solder, bonding wire, or the like.

The sealing member r 13 made of quartz glass has a contact portion with the package substrate 12 hermetically sealed by solder or the like.

As described above, the ultraviolet light LED package 10 is a package in which the LED die 11 is sealed only with an inorganic material.

The sealing member 13 made of quartz glass does not allow water vapor to pass therethrough, and the contact portion is hermetically sealed, thereby making it possible to prevent water vapor from reaching the LED die 11 and to prevent deterioration in the LED die 11 due to water vapor.

A light emission wavelength of the LED die 11 can be selected depending on a target (bacteria or viruses) to be sterilized or inactivated. As an example, it is possible to use the LED die 11 that emits ultraviolet light having a wavelength in the range of 100 to 280 nm, which has strong inactivation capability against bacteria or viruses.

### Mounting Substrate 20

The mounting substrate 20 is an insulating substrate made of a glass composite, provided with wiring including a via and an electrode. The mounting substrate 20 preferably has a reflective layer provided on the front surface thereof and configured to reflect ultraviolet light. For example, a solder resist ink having high reflectivity with respect to ultraviolet light is applied to the front surface of the mounting substrate.

One or more ultraviolet light LED packages 10 are mounted on the mounting substrate 20 and are mounted thereon by solder.

In the example illustrated in FIGS. 1 to 4, two ultraviolet light LED packages 10 are mounted on one mounting substrate 20 with a predetermined gap therebetween. The mounting substrate 20 of the present embodiment has a shape similar to that of the number 8, and the shape has approximately circular regions formed for two ultraviolet light LED packages 10 to be respectively mounted thereon and connected to each other by a connection region having a smaller diameter than those of the circular regions.

The wiring of the mounting substrate 20 is connected to the wiring of the package substrate 12 of the mounted ultraviolet light LED package 10.

The mounting substrate 20 is disposed in a recessed portion formed in an upper surface of the metal housing 30. The mounting substrate 20 is smaller than the upper surface of the metal housing 30, and an upper surface region of the metal housing 30, which is not covered by the mounting substrate 20, is present around the periphery of the mounting substrate 20.

### Housing 30

The housing 30 is a plate-shaped substrate configured for the mounting substrate 20 to be mounted thereon, and has a shallow recessed portion formed in the upper surface thereof and configured to accommodate the mounting substrate 20 therein. The shape of the recessed portion is formed along the outer shape of the peripheral edge of the mounting substrate 20. The housing 30 desirably has high thermal conductivity and rigidity. For example, the housing 30 is preferably formed of a metal having high thermal conductivity (for example, aluminum). Here, the aluminum die-cast molded housing 30 is used. Hereinafter, the housing 30 will be referred to as the metal housing 30.

It is noted that the housing 30 can also be formed of a resin (engineering plastic) containing a metallic filler such as aluminum or carbon. A polycarbonate, a polyamide, or the like can be used as a resin to be mixed with a filler.

The metal housing 30 has a convex rib 31 provided on the upper surface thereof and formed along the outer side of the peripheral edge of the recessed portion formed to accommodate the mounting substrate 20 therein. The cross-sectional shape of the rib 31 is almost a semicircle.

It is noted that the metal housing 30 has a hole (for example, a screw hole) 33 of the mounting substrate and formed for the fastening tool 60 to be fastened therethrough.

### Lens 40

The lens 40 is formed of a material through which ultraviolet light passes and includes a dome-shaped lens body 41 and a flange portion 42 formed to be integrated with a peripheral edge portion of the lens body 41. The lens body 41 is disposed on the mounting substrate 20 to cover the ultraviolet light LED package 10. Since the lens body 41 is formed to have a dome shape, a space is provided between the lens body 41 and the ultraviolet light LED package 10.

The flange portion 42 of the lens 40 covers a peripheral edge portion of the mounting substrate 20, and an upper surface of the metal housing 30 around the periphery of the mounting substrate 20.

Here, two dome-shaped lens bodies 41 are provided and respectively cover two ultraviolet light LED packages 10. The two dome-shaped lens bodies 41 are connected to each other by the flange portion 42. However, the shape of the lens 40 is not limited to this shape, and may be a shape in which one dome-shaped lens body 41 covers two ultraviolet light LED packages 10 and has the flange portion 42 provided around the periphery thereof.

The lens 40 is preferably formed of, for example, a resin through which ultraviolet light passes, such as a silicone resin, so as to ensure elasticity of the lens 40. Suitable elasticity is approximately Shore A 50.

Meanwhile, the lens 40 may be formed of a material having high thermal conductivity, such as glass. This configuration will be described in a first modification.

It is noted that the flange portion 42 is designed not to cover a position of the metal housing 30, where the hole (for example, screw hole) 33 formed in the metal housing 30 and provided for the fastening tool 60 is located.

### Cover 50

The cover 50 has an opening at the position thereof, corresponding to the position of the lens body 41, and is formed to have a shape to cover the upper surface of the flange portion 42 of the lens 40.

The cover 50 has a through-hole (for example, a screw hole) 51 formed for the fastening tool 60 to pass therethrough.

The cover 50 is preferably made of a material having high rigidity and high reflectivity with respect to ultraviolet light. For example, the die-cast molded aluminum cover 50 can be used. Further, it is also possible to use the cover 50 formed of a resin having high rigidity, for example, a resin (engineering plastic) containing a metal filler, such as aluminum or carbon.

### Fastening Tool 60

The fastening tool 60 presses the cover 50 against the metal housing 30 such that the cover 50 is fixed to the metal housing 30 in a state in which the flange portion 42 of the lens 40 is interposed between the cover 50 and the metal housing 30. For example, a screw can be used for the fastening tool 60.

### Rib 31

The convex rib 31 is provided on a region of the upper surface of the metal housing 30, which is in contact with the flange portion 42 of the lens 40, and is formed along the peripheral edge of the mounting substrate 20. A portion of the lower surface of the flange portion 42 of the lens 40, which is in contact with the rib 31, is pressed against the rib 31 by the cover 50 and is deformed such that a space in the lens 40 is physically sealed and is airtightly sealed. The lens 40 is fixedly deformed along the shape of the rib 31 due to elasticity thereof. Accordingly, the portion of the lens 40, which is pressed by the rib 31, can be sealed by obtaining a high sealing function, thereby making it possible to prevent water from infiltrating into the waterproof ultraviolet light-emitting module 1.

In this case, a peripheral edge 21 of the mounting substrate 20 is designed to be located outside an inner peripheral edge portion 43 of the lens body 41, which is located on the ultraviolet light LED package side, and is designed to be located inside the rib 31 of the metal housing 30. With this arrangement, the bottom surface of the lens body 41 and the lower surface of the flange portion 42, which are located between the inner peripheral edge portion 43 of the lens body 41 and a contact portion of the flange portion 42 with the rib 31, are in contact with the peripheral edge portion of the mounting substrate 20. Further, force through which the cover 50 is fastened to the flange portion 42 can be applied as force through which the peripheral edge portion of the mounting substrate 20 is pressed against the metal housing 30.

Accordingly, the mounting substrate 20 can be pressed against and fixed to the housing 30 without using a screw or the like.

### Step 44

Preferably, a step 44 is provided on the lower surface of the flange portion 42 of the lens 40 and is formed along the peripheral edge of the mounting substrate 20. A region inside the step 44 (a region from the step 44 to the edge portion 43) is in contact with the upper surface of the mounting substrate 20. A region outside the step 44 (a region from the step 44 to the outer periphery of the flange portion 42) is in contact with the upper surface of the metal substrate.

Accordingly, since the lower surface of the region inside the step 44 of the lens 40 (the region from the step 44 to the edge portion 43) can be brought into close contact with the upper surface of the mounting substrate 20, force through which the lens 40 presses the peripheral edge of the mounting substrate 20 against the metal housing 30 can be evenly applied to the mounting substrate 20, thereby more firmly fixing the mounting substrate 20.

In other words, the flange portion 42 of the elastic lens 40 has a surface ((the region from the step 44 to the edge portion 43) and adapted to press the mounting substrate 20, separately from a surface of the flange portion 42 (the region from the step 44 to the edge portion 43), which is in contact with the rib 31 to form a sealing surface, thereby pressing the mounting substrate 20 to suppress positional deviation of the mounting substrate 20 pressing the metal housing 30 with respect to the metal housing 30.

As described above, since a strong force is applied from the lens 40 to press the mounting substrate 20 against the metal housing 30, the entirety of the lower surface of the mounting substrate 20 can be brought into close contact with the metal housing 30. As a result, heat emitted from the ultraviolet light LED package 10 is conducted to the mounting substrate 20 and is spread throughout the entirety of the mounting substrate 20. Then, the heat is conducted from the entirety of the lower surface of the mounting substrate 20 to the metal housing 30, thereby enabling the heat to be dissipated to the outside from the lower surface of the metal housing 30.

### Thermally Conductive Sheet 70

A thermally conductive sheet 70 having insulation properties is interposed between the lower surface of the mounting substrate 20 and the metal housing 30. In this manner, the lower surface of the mounting substrate 20 can be electrically insulated from the metal housing 30, and simultaneously, thermal conductivity from the back surface of the mounting substrate 20 to the upper surface of the metal housing 30 can be further improved.

For example, a sheet made of silicone rubber can be used as the thermally conductive sheet 70.

### Power Supply Line 90

As illustrated in FIG. 4, the metal housing 30 and the thermally conductive sheet 70 have a first through-hole 32 and a second through-hole 71 formed to respectively pass therethrough in the thickness direction and provided at positions corresponding to each other (overlapping each other). A power supply line 90 is disposed to pass through the first through-hole 32 and the second through-hole 71. One end of the power supply line 90 is connected to the wiring on the lower surface of the mounting substrate 20, and the other end thereof is pulled out from the lower surface of the metal housing 30 to the outside. Accordingly, power is supplied to the ultraviolet light LED package 10 via the power supply line 90, thereby enabling the ultraviolet light LED package 10 to emit light.

The first through-hole 32 in the metal housing 30 is blocked by a filling member 80 and is waterproofed such that water droplets are prevented from infiltrating into the interior.

### Operation of Each Portion During Ultraviolet Light Emission

A description will be given as to a function of each portion in a case where ultraviolet light is emitted from the waterproof ultraviolet light-emitting module 1.

When power is supplied to the ultraviolet light LED package 10 from the power supply line 90, the internal LED die 11 emits ultraviolet light. The emitted ultraviolet light passes through the sealing member 13 made of quartz glass, passes through the lens body 41 of the lens 40, and is emitted to the outside.

The flange portion 42 of the lens 40 includes the step 44, and the flange portion 42 presses the mounting substrate 20 by force through which the fastening tool 60 fastens the cover 50. As a result, since positional deviation of the mounting substrate 20 can be prevented, the position of the sealing member 13 of the ultraviolet light LED package 10 on the mounting substrate 20 and the position of the lens body 41 can be determined, thereby preventing deterioration in light distribution. Furthermore, in a case where the flange portion 42 is not provided and the lens is bonded only to a region of the upper surface of the mounting substrate 20, the fixed state of the lens is not stable and is not desirable. Therefore, the flange portion 42 is formed over a region wider than the rib 31 of the metal housing 30 to prevent deterioration in light distribution and to improve water resistance.

Heat emitted from the LED die 11 is conducted to the package substrate 12 and is conducted to the mounting substrate 20. Thereafter, the heat is spread throughout the entirety of the mounting substrate 20. The heat is conducted from the entirety of the lower surface of the mounting substrate 20 through the thermally conductive sheet 70 to the upper surface of the metal housing 30. Since the metal housing 30 is formed of a metal having good thermal conductivity, the heat is dissipated from the metal housing 30 to the outside.

Accordingly, since the heat generated by the LED die 11 can be efficiently dissipated through the metal housing 30, it is possible to improve the lifespan of the LED die 11, and to suppress deterioration in the ultraviolet light LED package 10.

Even if an apparatus having the waterproof ultraviolet light-emitting module 1 disposed therein is exposed to water droplets or placed in a high-humidity environment, water droplets do not enter the inside of the lens 40 because the flange portion 42 is pressed against the upper surface of the metal housing 30 and the rib 31 by the cover 50 at a contact portion between the flange portion 42 of the lens 40 and the metal housing 30. Furthermore, since the first through-hole 32 is filled with the filling member 80, water droplets do not enter the inside.

Even if the lens 40 is made of a resin, water droplets cannot pass through the lens 40, thereby preventing water droplets from infiltrating into the space in the lens 40 (FIG. 6).

In a case where the lens 40 is made of a resin, water vapor passes through the lens 40 and enters the space between the lens body 41 and the ultraviolet light LED package 10. However, since the sealing member 13 of the ultraviolet light LED package 10 is made of quartz glass, water vapor cannot enter the inside of the sealing member 13.

As a result, since moisture (water droplets and water vapor) does not reach the LED die 11 of the ultraviolet light LED package 10, deterioration in the LED die 11 can be prevented.

In this manner, the waterproof ultraviolet light-emitting module 1 achieves waterproof functionality by sealing the LED die 11 with two stages by the ultraviolet light LED package 10 in which the LED die 11 is sealed only with an inorganic material, and the metal housing 30 and lens 40.

In the waterproof ultraviolet light-emitting module 1 of the present embodiment is configured such that the cover 50 is disposed at a position spaced apart from a light distribution portion (the lens body 41 of the lens 40), and the flange portion 42 of the lens 40 is fastened to the metal housing 30 by the cover 50. Accordingly, a degree of freedom of design of the lens shape of the light distribution portion (the lens body 41) is high.

Furthermore, ultraviolet light that has been guided through the lens body 41 and reached the flange portion 42 can be reflected by the inner wall surface of the cover 50, which is in contact with the flange portion 42, and can be emitted directly to the outside. Among the light reflected by the inner wall surface of the cover 50, light oriented toward the front surface of the mounting substrate 20 can be reflected by a light-reflecting layer disposed on the upper surface of the mounting substrate 20 and can be emitted to the outside, thereby enabling the light to be reused.

Furthermore, since the entirety of the flange portion 42 of the lens 40 is pressed by the frame-shaped cover 50 having rigidity, force is applied to the entirety of the flange portion 42 and as such, deviation of the lens 40 is less likely to occur.

At the same time, repulsive force generated by elasticity of the lens 40 prevents loosening of a screw serving as the fastening tool 60 with the same effect as a spring washer.

### Assembly Procedure of Each Portion During Ultraviolet Light Emission

A description will be given as to an assembly procedure of the waterproof ultraviolet light-emitting module 1 using FIGS. 4 and 7.

As illustrated in FIG. 4, the ultraviolet light LED package 10 is mounted on the mounting substrate 20.

The thermally conductive sheet 70 is disposed on the metal housing 30, and the mounting substrate 20 is mounted thereon. The lens 40 is placed on the metal housing 30, and then, the cover 50 is placed thereon. As illustrated in FIG. 7, the fastening tool (screw) 60 is screwed into the screw hole 33 in the metal housing 30 through the through-hole 51 in the cover 50.

As described above, the waterproof ultraviolet light-emitting module 1 can be manufactured.

### First Modification

A waterproof ultraviolet light-emitting module of a first modification will be described.

In the above-described embodiment, the lens 40 is formed of a resin allowing ultraviolet light to pass therethrough, and the flange portion 42 of the lens 40 is interposed between the cover 50 and the metal housing 30 to seal the lens 40. However, the lens 40 may also be formed of a non-elastic material such as glass.

When the lens 40 is formed of glass or the like, a resilient sealing member is prepared and disposed on a portion at which the flange portion 42 is in contact with the upper surfaces of the metal housing 30 and the mounting substrate 20. In this manner, the sealing of the lens 40 can be achieved, similarly to the embodiment.

Preferably, the sealing member is a resin resistant to deterioration due to ultraviolet light, such as a silicone resin.

### Second Modification

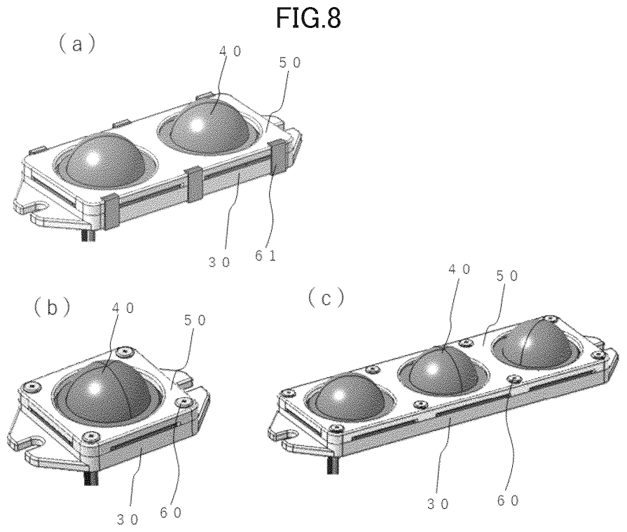

A waterproof ultraviolet light-emitting module of a second modification will be described using FIG. 8(a).

The modification in FIG. 8(a) is an example in which a hook 61 is used as the fastening tool 60 of the waterproof ultraviolet light-emitting module. Another structure such as the hook 61 can be used as the fastening tool 60 as long as the structure is capable of pressing the cover 50 against the metal housing 30.

### Third Modification

A waterproof ultraviolet light-emitting module of a third modification will be described using FIGS. 8(b) and 8(c).

The modification in FIG. 8(b) is a structure in which only one ultraviolet light LED package 10 of the waterproof ultraviolet light-emitting module is mounted on the mounting substrate 20.

The modification in FIG. 8(c) is a structure in which three ultraviolet light LED packages 10 of the waterproof ultraviolet light-emitting module are mounted on the mounting substrate 20.

It should be noted that four or more ultraviolet light LED packages 10 can also be mounted on the mounting substrate 20.

Furthermore, the lens body 41 of the lens 40 is not limited to a structure in which one lens body 41 is disposed for one ultraviolet light LED package 10, and the lens body 41 may be disposed to cover a plurality of ultraviolet light LED packages 10.

### Fourth Modification

A waterproof ultraviolet light-emitting module of a fourth modification will now be described.

In the above-described embodiment, although a hemispherical shape is illustrated as the shape of the lens body 41 of the lens 40, the shape of the lens body is not limited to the hemispherical shape, and a desired shape can be designed to obtain a desired light distribution pattern of ultraviolet light. For example, the lens 40 can be formed to have a convex-and-concave shape to implement narrow-angle or wide-angle light distribution.

### INDUSTRIAL APPLICABILITY

The waterproof ultraviolet light-emitting module of the present embodiment and the modifications can be used in an ultraviolet light sterilization apparatus configured to irradiate a portion to which mold, bacteria, viruses, and the like are attached with ultraviolet light and to sterilize the portion. For example, the waterproof ultraviolet light-emitting module can be mounted in a car air conditioner, a humidifier, a rice cooker, a microwave oven, a dishwasher, an electric pot, a refrigerator/freezer, a sterilization chamber, a facial apparatus, an indoor/outdoor ultraviolet light irradiator, and the like.

The waterproof ultraviolet light-emitting module is used as an ultraviolet light source module mounted in a sterilization apparatus for fluids such as water and gas. For example, the waterproof ultraviolet light-emitting module can be used as an ultraviolet light source module configured to irradiate, with ultraviolet light, and sterilize water stored in a storage tank of an ice maker, water in a water pipe or a water heater, drinking water of a water dispenser, cooling water of a circulation apparatus (chiller), a beverage in a drink dispenser, and the like.

### REFERENCE SIGNS LIST

1: ultraviolet light-emitting module
10: ultraviolet light source (ultraviolet light LED package)
11: die
12: package substrate
13: sealing member
20: mounting substrate
21: peripheral edge
30: metal housing
31: rib
33: screw hole
40: lens
41: lens body
42: flange portion
43: edge portion
44: step
50: cover
51: through-hole
60: fastening tool
61: hook
70: thermally conductive sheet
80: filling member
90: power supply line

1. A waterproof ultraviolet light-emitting module comprising an ultraviolet light source, a mounting substrate, a housing, a lens, a cover, and a fastening tool, wherein
the ultraviolet light source is an LED package configured such that an LED die emitting ultraviolet light is enclosed in a package,
the mounting substrate includes wiring, is configured such that the ultraviolet light source is mounted thereon, and is disposed on the housing,
the housing has a plate shape allowing the mounting substrate to be mounted thereon,
the lens is formed of a material allowing the ultraviolet light to pass therethrough and includes a lens body and a flange portion, the lens body having a dome shape and being disposed to cover the ultraviolet light source with a space interposed therebetween, the flange portion being provided to be integrated with a peripheral edge portion of the lens body,
the cover covers the flange portion of the lens,
the fastening tool presses the cover against the housing such that the cover is fixed to the housing in a state in which the flange portion of the lens is interposed between the cover and the housing,
the housing has a convex rib provided on a portion thereof in contact with the flange portion of the lens, the convex rib being formed along a peripheral edge of the mounting substrate, and
a portion of a lower surface of the flange portion of the lens, the portion being in contact with the rib, is deformed by being pressed against the rib by the cover such that the space in the lens is sealed.

2. The waterproof ultraviolet light-emitting module according to claim 1, wherein a material forming the lens is a resin.

3. The waterproof ultraviolet light-emitting module according to claim 1, wherein the peripheral edge of the mounting substrate is located outside an inner peripheral edge portion of the lens body, the inner peripheral edge portion being in contact with the space, and is located inside the rib of the housing.

4. The waterproof ultraviolet light-emitting module according to claim 3, wherein
the lower surface of the flange portion of the lens has a step at the peripheral edge of the mounting substrate,
a region of the lower surface of the flange portion of the lens, located inside the step, is in contact with an upper surface of the mounting substrate, and
a region of the lower surface of the flange portion of the lens, located outside the step, is in contact with an upper surface of the housing.

5. The waterproof ultraviolet light-emitting module according to claim 1, wherein a thermally conductive sheet having insulating properties is interposed between a lower surface of the mounting substrate and the housing.

6. The waterproof ultraviolet light-emitting module according to claim 1, wherein the fastening tool is a screw.

7. The waterproof ultraviolet light-emitting module according to claim 2, wherein
the ultraviolet light source includes a substrate configured for the LED die to be mounted thereon and a sealing member transparent to the ultraviolet light, the sealing member being configured to cover a space in which the LED die on the mounting substrate is disposed, and
the sealing member is glass.

8. The waterproof ultraviolet light-emitting module according to claim 5, further comprising a power supply line and a filling member, wherein
the housing and the thermally conductive sheet have a first through-hole and a second through-hole formed to respectively pass therethrough in a thickness direction of the housing and the thermally conductive sheet, the first through-hole and the second through-hole being provided at positions corresponding to each other,
the power supply line passes through the first through-hole and the second through-hole, one end of the power supply line is connected to the wiring of the mounting substrate, and the other end of the power supply line is pulled out to an outside of the housing, and
the first through-hole in the housing is filled with the filling member such that the housing is waterproofed.

9. The waterproof ultraviolet light-emitting module according to claim 3, wherein
two or more of the ultraviolet light sources are mounted on the mounting substrate with a gap therebetween, and
the rib of the housing and the flange portion of the lens are provided along the peripheral edge of the mounting substrate configured for the two or more ultraviolet light sources to be mounted thereon.

10. The waterproof ultraviolet light-emitting module according to claim 1, wherein the housing is made of a metal or a resin containing a metal filler.
